(19) United States
(12) Patent Application Publication
(10) Pub. No.: US 2002/0096543 A1
(43) Pub. Date: Jul. 25, 2002

(54) Device for monitoring the administration of doses and system for monitoring the administration of doses

(76) Inventor: Raimo Juselius, Vihti (FI)

Correspondence Address: SWIDLER BERLIN SHEREFF FRIEDMAN, LLP, 3000 K STREET, NW, BOX IP, WASHINGTON, DC, 20007, US

(21) Appl. No.: 10/051,123

(22) Filed: Jan. 22, 2002

Related U.S. Application Data
(60) Provisional application No. 60/262,418, filed on Jan. 19, 2001.

(51) Int. Cl. B05B 11/02

(52) U.S. Cl. 222/631

(57) ABSTRACT

The invention relates to a portable control device for monitoring the administration of doses by a diabetic. The control device is arranged in conjunction with a portable dosage unit having a body and an injection member designed to deliver the dose to the diabetic and attached to the body of the portable dosage unit. The dosage unit has a removable cap over the injection member. The control device is provided in the cap and is movable together with the cap. The control device comprises a sensor arranged to detect the removal of the cap from over the injection member and connected to an indicator of the control device for showing if the cap has been removed from over the injection member for administering insulin.

### REFERENCES TO EARLIER APPLICATIONS

The present application is based on U.S. provisional application No. 60/262,418, filed on Jan. 19, 2001.

### FIELD OF THE INVENTION

The invention relates to a device for monitoring the administration of doses by a diabetic. The invention also relates to a system for monitoring the administration of doses by a diabetic.

### BACKGROUND OF THE INVENTION

It is known to use various medicament containers with alarm systems based on timers for drug doses which are to be administered at intervals and are usually in the form of pills, tablets, or corresponding doses to be administered orally, and as an example it is possible to mention the alarm systems for medicament containers disclosed in the application publications GB 2179919 and FR 2666225.

Diabetes is a rapidly increasing serious metabolic disorder that is caused by lack of insulin as a result of decayed pancreatic islet cells (type 1, i.e. juvenile-onset diabetes mellitus) or by the fact that insulin trasmittors have become “lazy” (type 2, i.e. adult-onset diabetes). When untreated, both types of diabetes will cause an increase in the sugar content of blood, coma and death.

In principle, there exists a very simple medicament for the treatment of diabetes: the missing insulin is injected from outside the body. In practice, the treatment requires precision and care, because the amount of insulin in the blood circulation has to be correct for the situation in question. The correct amount depends (primarily) on the sugar-forming substances one has eaten as well as on the amount of physical exercise.

Too small temporary amount of insulin increases the sugar content of blood to an overly high level, and too large amount of insulin results in too low blood sugar level (insulin shock). Thus, a diabetic must constantly balance between two inconvenient phenomena. Of these two the too low sugar content, i.e. insulin shock is, however, more dangerous in the short run.

During the years, poor treatment (=poor treatment balance), i.e. constant overly high blood sugar content, may cause severe disturbances in peripheral blood circulation, even amputations, weakening of eye sight, difficult skin symptoms, kidney failure, and other serious secondary diseases.

Normally, a diabetic person has three different injection pens: a morning insulin pen containing typically a mixture of slowly acting insulin and rapidly acting insulin (e.g. Mixtard), a daytime pen containing rapidly acting insulin (e.g. Actrapid, Novorapid), which is typically used in connection with large meals, as well as a so-called evening pen, typically containing slowly acting insulin (e.g. Protaphan). Different types of insulin should be stored—at least—in pens of different colour, preferably even in pens of different manufacturers, because the wrong type of insulin at the wrong time is harmful, to say the least, and in the worst case even lethal.

Constant control constitutes an important part in the treatment of diabetes. The types of insulin and the injection times as well as the measured blood sugar values are marked in a control book. By comparing these values—together with a doctor and a diabetes nurse—to a so-called long-term blood sugar value measured in a laboratory in periods of few months, it is possible to develop the treatment so that it better corresponds to the way of life of the diabetic in question.

Temporary blood sugar content can nowadays be easily checked by each diabetic himself/herself by means of a so-called blood sugar indicator from a blood sample taken from the tip of a finger. The indicators are relatively cheap, but disposable strips are significantly expensive.

Because so-called long-term insulin is always injected “beforehand”, in a way, it is essential that the diabetic remembers to take care of his/her carbohydrate intake regularly, i.e. in practice, a portion of food containing an amount of carbohydrates corresponding to the situation in question should be eaten every couple of hours.

During the treatment period all diabetics are taught the carbohydrate contents of different nutrients, and for home care there are so-called “conversion tables” which show the average carbohydrate content of nutrients. A scale, the afore-mentioned table, a predetermined meal plan, and the tables in packages containing information on the nutrients become familiar to all diabetics.

An accustomed diabetic does not necessarily need said tables or scales, but he is able to estimate the carbohydrates on the basis of his/her experience with sufficient reliability.

It is a common misconception that a diabetic must not eat sugar. Diabetes is not “allergy to sugar”, but actually quite the opposite: diabetics must eat sugar-forming substances, and even quite regularly, and the correct amount at the correct time.

On an average, a diabetic must perform a treatment action related to the diabetes (snack, insulin injection, measuring of blood sugar) every couple of hours during the entire awake time. When changes occur in the normal way of life, for example a considerable amount of physical exercise is taken, it is necessary to perform such actions even more often. After a day with a large amount of physical exercise, it is necessary to measure the blood sugar in the middle of the night as well. In other words, the life of a diabetic is rather regular and seems to be quite pre-programmed. But it is possible to depart from the routines, as long as you know what you are doing.

Thus, the treatment of diabetes primarily requires administration of fixed doses of insulin at predetermined times according to the treatment plan. Consequently, devices have been developed for taking insulin doses by means of a syringe.

U.S. Pat. No. 4,950,246 discloses a syringe intended for the use of a person having diabetes, an “injection pen”, which can be used to meter a predetermined dose of insulin. The syringe has an integrated system with a sensor monitoring the progression of a pump rod inside the syringe and giving information to an electronic control unit for administering a correct dosage at the time of injection. In this syringe, the only alarm is an indication on the emptying of the reservoir to be expected.

The international publication WO 99/43283 discloses a new regimen for the treatment of diabetes, which can be easily implemented by the equipment available without making structural changes in the injection pen itself. This is implemented by means of special stand that functions as a control device for doses and contains several holes, “sockets” for the injection pens containing insulin. The stand comprises recesses for placing the injection pens in an erect position therein. For each injection pen there is a pair of indicators. The device is arranged to give an alarm in a certain order, wherein the indicator of the respective injection pen gives an alarm that it is time to administer a dose from the injection pen in question. The act of removing this injection pen from the stand is detected by means of a suitable sensor detecting the movement of the injection pen away from the stand. When the removal of the injection pen has been detected, a second indicator is shifted to a state in which it indicates that the dose has been administered. The indication may take place visually, for example by means of a signal light. The publication discloses how the injection pen can be locked to the stand at other times to prevent inappropriate use of the same. The diameters of the pens determine the size of the sockets in such stands. In this publication, an indicating device attachable to an injection pen is also described. This device is arranged to detect the movement of a push-button at the end of the opposite to the needle. The device interpretes the down movement of the push-button operating the piston as the act of administering the insulin dose from the pen. The detection is based on mechanical contact (micro-switch) between the device and the push-button.

### SUMMARY OF THE INVENTION

It is a purpose of the present invention to provide a system, which enables the monitoring of the administration of insulin doses 24 hours a day, and by means of which it is possible to implement home care of diabetics. The monitoring system especially enables the multi-injection treatment of working people who must leave home on a regular basis and who can not take the device with them. The invention enables multi-injection treatment both at home and in work in such a manner that a doctor or another outside expert can constantly monitor and control the treatment at least on a daily basis. The invention is especially well suited for self-care of the diabetic and learning of the self-care, and it is also possible to enhance patient compliance thereby.

A control device that can be easily mounted on any injection pen and can be carried by the person who must regularly administer the doses improves the applicability of the concept of a multi-injection care of diabetes that can be monitored and controlled by the person himself/herself and outside experts and care personnel, even if the person is not at home at all times. Prevous attempts to provide a simple control device have proven insufficient, because it has required either changes in the internal construction of the injection pen or mounting of mechanical contacts. The invention resolves the problem in a simple manner. The control device is mounted on the cap covering the needle of an injection pen and its removal form the body of the pen can be detected, based on the principle that when the cap is removed, the physical surroundings of the cap and device on it change because the injection member attached to the body, such as a needle, will be absent from the inside of the cap. If the cap is away a sufficient long time, the control device inerpretes this as an injection (administering the dose). The detection takes place in a contactless manner. A sensor complementary to the detection sensor in the control device can be attached to the body, for example at the base of the needle.

A central part of the monitoring system is a stand-like control device which is located at the diabetic's home and contains data processing and memory capacity and can be programmed, i.e it is a console containing at the same time locations for accomodating several injection pens. It is an aim of the control device to act as a “personal digital diabetes nurse” for every diabetic. Although the device was initially designed for children and elderly people, it facilitates the life of any “independent” diabetic and guides to the correct treatment balance. The basic idea behind the console is to provide an active holder for injection pens (insulin syringes), a snack and treatment reminder as well as “a storage” for treatment data and an information source on nutrition in such a manner that the instructions given by the device adapt to the changes in the daily routine. The instructions given by it can be changed so that they better correspond to the weekly routine of the person in question.

The control device is pre-programmed with co-operation of the diabetes doctor and/or diabetes nurse and a nutrition therapist in such a manner that it corresponds to the average daily routine of the diabetic in question as closely as possible. The device reminds the patient of snacks, insulin injections and blood sugar measurements according to a predetermined schedule. It is possible that the device contains a programmable calendar of at least a short-time span in which different dates have information of their own for example on the size of the insulin doses. Preferably such a calendar repeats itself in periods of one week if the diabetic follows a set weekly routine. In that case, each day of the week may have pre-programmed instructions of its own but this data can be reprogrammed if it is later discovered that some instructions need altering (for example the size of the doses) either due to a changed weekly routine or for other reasons.

The device can be taken in use already in the hospital on the first day of the treatment of diabetes, it can be programmed and the program can be changed during the treatment, and when it is time to return home, the device is taken along to support home care.

In home care the device is connected through a telephone network to a computer of an expert, for example the doctor responsible for the treatment, and the doctor can familiarize himself/herself with the treatment history on the basis of the insulin amounts, injection times and measured blood sugar information, and change the treatment instructions in real time by remote control via a data transmission line (e.g. telephone network), if necessary. The doctor can also discuss the treatment results with the diabetic and make an agreement on changes before making them via remote control.

The control device is also based on the fact that each action reminded upon by the control device must be acknowledged either as performed or postponed (wherein the device reminds the user on the undone treatment action within a fixed period of time from the first reminder). According to the principle described hereinbelow the administration of the injection is automatically registered with certain conditions, whereas certain actions related to the treatment must be acknowledged via a user interface by using buttons, etc. The device can give a warning with a special warning signal if the diabetic is about to make a mistake, especially a so-called double injection (another injection within a short time due to forgetfulness). The device can also be programmed in such a manner that it sends an automatic alarm to a desired place (for example to a relative of the diabetic, or to an emergency exchange) if the actions are not acknowledged as performed within a predetermined period of time.

These automatic warning and alarm functions of the control device substantially increase safety. The device prevents for example a double injection in such a manner that after the injection the injection pen is lowered down to such a position that it can be taken out only by pressing the buttons on the user interface. Even an accustomed “independent” diabetic may sometimes forget either to take the insulin injection or—even worse—that he has already taken the injection, and thus injects himself/herself twice (double injection, which can be fatal).

The function also prevents the injection of a wrong type of insulin, because only the correct pen is available at the correct time. The control device is advantageously provided with a dialog function before each insulin injection, it is, for example, possible to input information in the control device on earlier physical exercises as well as on the probable amount of exercise in the period of next two hours, and the device can thus give a recommendation on the insulin dose and/or snack amounts depending on the programming. In that case the device has computing capacity.

On the other hand, the device can contain pre-entered information on the weekly schedule, and before each injection, the control device can display default information on such actions of the diabetic on just the day of the week and injection moment in question in the weekly schedule which have either been performed or should be performed after taking the dose and which affect the insulin dosage, for example: “day: Wednesday, dosage: morning insulin; action: going swimming?”. By means of the buttons in the user interface the diabetic can confirm these pieces of default information as correct or incorrect, wherein the device can determine the most suitable values on the basis of the changed information.

An important additional feature of the console-type control device is that it is designed to receive information from the device that is on the portable pen. The control device of the portable pen can be charged by the console with simulataneous change of information between the console and the control device of the portable pen.

### BRIEF DESCRIPTION OF THE DRAWINGS

In the following, the invention will be described in more detail with reference to the appended drawings, in which

FIG. 1 shows a side-view of the control device according to the invention,

FIG. 2 shows top view of the control device of FIG. 1,

FIG. 3 shows the operation of the control device in a situation where the indicator indicates an event of administration of a dose,

FIG. 4 shows a way of using the sockets of the control device,

FIG. 5 shows the charging of a portable device,

FIG. 6 shows the principle of recognizing an event of administration of a dose in a portable injection pen,

FIG. 7 shows another embodiment of the portable injection pen,

FIG. 8 shows combining of blood sugar measurement with the control device, and

FIG. 9 shows a chart of the monitoring system in its entirety.

### DETAILED DESCRIPTION OF THE PREFERRED EMBODIMENTS

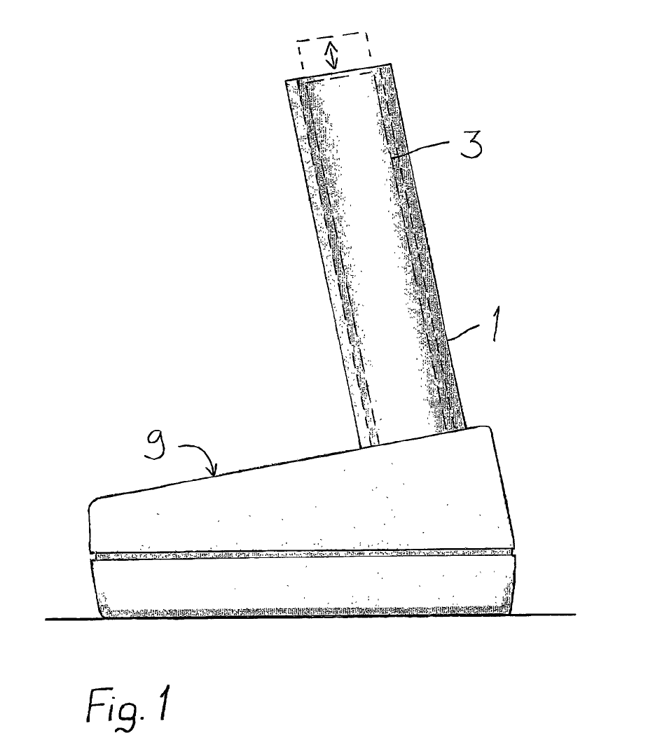

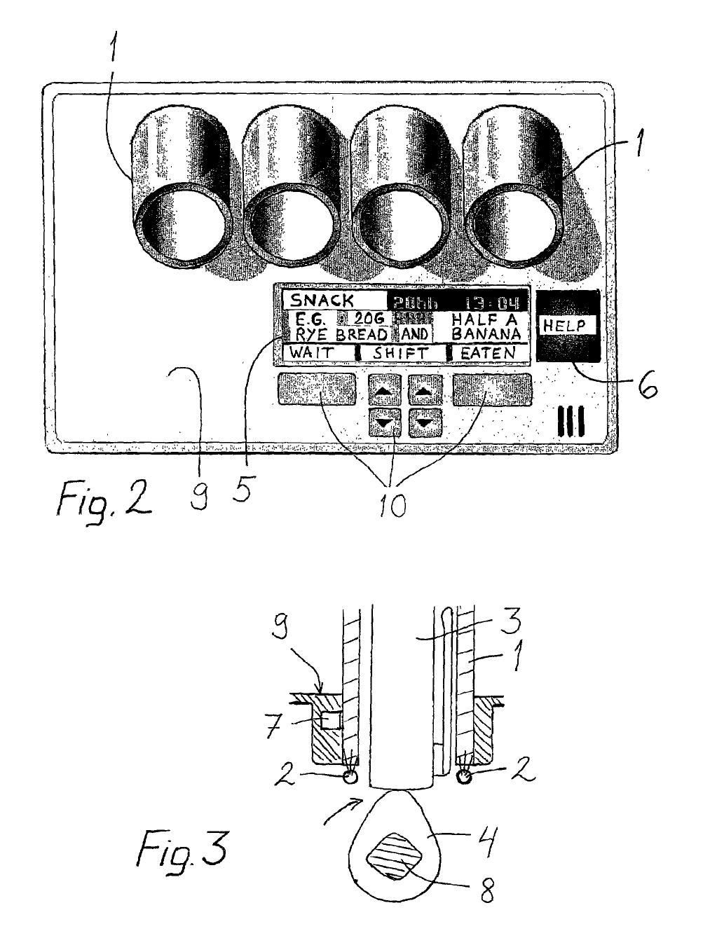

FIGS. 1 and 2 show a control device for doses which forms a stand for two or more medicament dosage units 3, in this case injection pens. The stand is intended to be placed for example on a table and hereinbelow the term “table control device” or “console” will also be used for the same. The stand is an electronic device whose data processing unit contains a programmable processor and memory capacity for storing information, as well as a clock device i.e. time measuring device that constitutes a timer. It is the purpose of the stand to control and monitor the administration of several doses during the day. To each injection pen, there is allotted a time of the day in the memory of the device telling when the injection should be taken. When it is the time to administer a dose from the pen, a visual indicator by the pen shows that the dose should be taken from the pen in question. At the same time an acoustic alarm can be given. The removal of the pen is detected by means of a suitable sensor, for example a sensor operating by means of a mechanical switch or on contactless principle, and when the pen is removed for a certain period of time, this is interpreted as an event of administration of the dose. When the event of administration of the dose has been registered in this way, the visual indicator by the pen remains, for a given time, for example until the beginning of the next standby time of the same pen, in a state that shows that the dose has already been taken from the pen. This is indicated advantageously with a colour light that can be easily detected. The indicator for the pen showing that a dose should be taken and the indicator showing that the dose has been taken can be different indicators which are turned “on” and “off”. It is, of course, possible to use physically the same indicator which changes its state, e.g. colour, according to the state of the pen in question. The operating principle is the same for each injection pen to be monitored in the stand. The injection pens can be different in that they contain medicaments which act differently and which should be taken at a certain time of the day. Especially in the treatment of diabetes, the pens may contain different types of insulin.

In the following the structure of the control device implementing the aforementioned operating principle will be described in more detail. FIG. 1 shows a side-view of a stand for keeping several dosage units 3, in this case injection pens containing insulin. The stand is a casing, provided with the necessary electronics therein and containing a coupling for an external power source (coupling for a battery charger or a plug to be inserted to a socket). For each injection pen there is a socket 1 to which it can be placed to a vertical position, or, as shown in the figure, to a sloping position. The socket 1 is composed of a tube, which is dimensioned in such a manner that the dosage unit 3 illustrated with broken lines is in the normal position located entirely in the tube, i.e. the end of the same is located inside the inlet opening of the tube so that it cannot be removed from the tube with fingers. The tube is attached to the body casing of the control device, to a recess located in the inclined front wall, i.e. the top wall 9 of the casing. The tube is made of transparent material, for example acrylic plastic, wherein it is easy to see whether there is an injection pen inside the tube.

In the same wall of the casing from which the tubes protrude, there is also a user interface, i.e. a display and buttons required for operating the device. The function of the display and buttons will be described hereinbelow.

FIG. 2 shows a top view of the device. As can be seen in the figure, the device contains tubes for four injection pens. The invention is not, however, restricted to the number of tubes, and thus, it also falls within the scope of the invention that there is only one tube for one injection pen, although the device is best suited for controlling the administration of different types of doses, wherein it is necessary to use at least two different dosage units 3, and at least two different sockets 1, respectively, for example when two different types of insulin are used for the treatment of diabetes. When the device contains two or more sockets 1, they are identical in shape so that a conventional injection pen fits in each socket. The front wall of the container, i.e. the top wall 9 which is slightly inclined, contains a display 5 on which different kind of information can be shown depending on the ways in which the device is programmed. In the normal state the display may only show for example the date and the time. For the treatment of diabetes a separate time has been programmed for each injection pen in the data processing unit of the device. The time measured by the time measuring device of the device is shown on the display in the manner mentioned above, and the data processing unit is arranged to compare the pre-programmed time with the time of the time measuring device. When it is time to administer a dose from the injection pen in question, the data processing unit gives commands to members which control the following functions: the alarm of the device gives a visual alarm by means of a signal light visible by the appropriate injection pen, and possibly an acoustic alarm as well. At the same time the display shifts to a state in which information programmed in the memory of the data processing unit is transmitted thereon, said information relating to the event of administration of that particular dose, for example the size of the dose (in the case of diabetes, units of insulin from the injection pen indicated by the alarm, or another measurement which is used in the injection pen and can be set therein before the injection). Thus, the data processing unit and the time measuring device cooperate in a manner similar to a timer.

Furthermore, it is possible that alarms relating to other actions can also be programmed in the data processing unit, which alarms are given at fixed times of the day, i.e. when the device gives an alarm, it does not necessarily indicate that a dose must be taken, but it can refer to other measures (meal, exercise, etc.) relating to the treatment of diabetes. The alarm of the device can in this case give a visually and/or acoustically different alarm than the alarm relating to the administration of a dose.

According to the principle disclosed in the publication WO 99/43283, the entire disclosure of which is incorporated herein by reference, the device also registers the removal of the dosage unit, and thus, the corresponding indicator will continuously indicate that the dose has been taken. This function will be described in more detail hereinbelow. If the dosage unit has not been removed within a fixed period of time after the alarm, the device gives another alarm.

The device can communicate with an outside supervisor for example by means of landline or wireless communication. Thus, for example a doctor can monitor the treatment, and in the case of a bilateral connection, the doctor can also program the device from a distance. Thus, it is possible to program different kinds of instructions in the data processing unit of the device, such as the sizes of doses, other instructions such as instructions for meals and exercises which can be shown on the display, etc. Furthermore, it is possible to change the administration times (set new alarm times) from a distance. Although the connection is protected as well as possible, to be sure the device contains a security function allowing only a change of particular scale one way or the other in the values relating to the administration of the medicament dose (time of day, size of the dose), i.e. an upper limit is determined for the changes (for example units of insulin and h). A special alarm may be automatically transmitted outside via the data transmission line if the device has not registered an event of administration of the dose within a predetermined time after the first alarm given to the user of the device. It is possible to provide the device with a function by means of which the user can avoid false alarm beforehand by entering information by means of the buttons of the user interface that certain injection will be taken care of in another way. Furthermore, the figure shows an emergency button 6, which, when pressed continuously for a predetermined period of time, for example 0.5 seconds, sends a special alarm outside. The special alarm may be programmed to be transmitted via the data transmission line directly to a special address, for example to an emergency centre. Furthermore, FIG. 2 shows function buttons 10, by means of which it is possible to move in the menu shown on the display 5 and confirm that different activities (meal, exercise) have been performed. These confirmations are also registered in the memory and the person supervising the treatment can monitor them via the data transmission line.

FIG. 3 shows schematically how the dosage unit 3 located inside the tube is lifted up to the operational position. The lower end of the dosage unit is positioned in the space below the top wall 9, the tube extending to this space as well. When the pre-programmed time and the time of the time measuring device match, an alarm relating to the event of administration of the dose is given, and in connection with the alarm the data processing unit also gives an actuating command to a lifter 4 located in the lower end of the tube and touching the lower end of the injection pen, said lifter lifting the pen inside the tube so that the opposite end of the pen rises above the inlet opening of the tube. In FIG. 3 the lifter 4 is implemented by means of an eccentric attached to an electrically rotated shaft 8 with a quadratic cross-section. The lifter 4 is of such a type that it does not lock the pen in its place, but the lower end of the pen rests freely on top of said lifter, and the pen can be removed by tilting the container, wherein the pen slides out, or by removing the tube. In a device containing several injection pens the eccentrics are placed on the same shaft 8 at regular angular distances so that they face different directions, and the shaft always revolves a corresponding distance (i.e. in a device with four sockets at distances of 90°) Furthermore, the visual light indicator 2 connected to the dosage unit is placed inside the casing, in the lower end of the tube in such a manner that it directs light from the end of the tube to the wall of the tube. Thus, the tube functions as a light guide and conducts the light emitted by the indicator into view, wherein it can be seen at least in the upper end of the tube as a glowing, ring-like light. The indicator 2 can be composed for example of a series of light-emitting diodes located at the end of the tube. When the event of administration of the dose has been registered, the colour of the light-emitting diodes can change (for example from green to red), or other light-emitting diodes that are placed for this purpose below the tube and emit different kind of light are switched on. Furthermore, a text may appear on the display 5 indicating from which socket 1 (tube) the dosage unit 3 should be taken and the size of the respective dose, and for this purpose, the front wall may contain a letter or a number by each tube.

When the lifter 4 has lifted the injection pen to the upper position, the removal of the same is detected by means of a sensor, for example with a sensor marked with the reference numeral 7 in FIG. 3, such as a light cell, operating on the contactless principle, and thus the indicator 2 indicates that the dose has been taken and the time (date and time of day) the dose was taken is at the same time registered in the memory of the data processing unit of the device. By means of the above-mentioned bi-directional data transmission connection, the person supervising the treatment, such as a doctor, can monitor the times of administration of the doses. The injection pen must be removed for a predetermined period of time, so that mere lifting of the pen up and lowering it back down right thereafter would not be registered as an event of administration of the dose.

The lifter 4 can be of another type as well, for example a plunger operating electrically by means of a magnetic coil, or by means of a motor of its own, in which case the lifters of different sockets are not mechanically connected to each other. If it is desired that the injection pen can be removed at other times than the time set for administrating the dose, the lifter 4 can also be arranged to operate manually, for example mechanically by means of a control apparatus, or with a special press button. The tubes surrounding the injection pens may also be easily detachable from the casing, for example they can be screwed off. If the tube is fixed to the body casing by means of a screw thread, the outwards protruding portion of the tube can then be handily adjusted as well. In practice, however, it has been observed that the insulin injection pens of different manufacturers differ from each other very slightly in length, whereas in diameters the variation is greater.

The function of the lifters 4 is advantageously arranged in such a manner that when the indicator 2 has detected that the dose has been taken, the lifter 4 of the corresponding socket is lowered down immediately or relatively soon after a delay time. This ensures the prevention of double injection, because the pen moves into the lower position after the dose has been taken, and it cannot be easily removed. Thus, in a situation when it is not the time to take the injection, all pens are in the lower position. When the lifter is implemented using eccentrics and a common shaft, the shaft must always, after an event of administration of a dose, revolve to such a position in which none of the eccentrics lifts the pen upwards, i.e. a distance half of the angular distance between two successive eccentrics. Thus, the mechanical solution itself, bringing forth clearly the pens from which a dose is intended to be taken while the other pens lie in their rest position, already ensures the prevention of double injection. Despite of this prevention of double injection provided already by the structure, it is possible to arrange an alarm within a fixed, predetermined precautionary time (for example within 2 hours from the administration), if, however, some kind of an attempt is made to remove the same pen. In the case of a double injection attempt, the alarm of the device receives information from the sensor 7 detecting the removal of the pen, and the alarm can give an acoustically or visually different alarm (warning signal) when compared to the normal alarm relating to the administration of a dose (reminder signal), thus warning of the risk of double injection. Furthermore, to be on the safe side, it is possible to provide the device with a function by means of which the special alarm is transmitted outside via a data transmission line when the device registers an injection from the same pen twice within the precautionary time, i.e. the sensor 7 has detected that within a set time (precautionary time) from the first registered injection, the same pen has been away for a fixed delay time, which is interpreted as a double injection. There are, however, cases in which the diabetic intentionally wishes to take another dose smaller than the normal dose from the same pen within a short period of time, for example due to an extra meal. This would be interpreted as a double injection. Unnecessary alarm can be avoided if it is possible to enter information in the device in which the user indicates that he is intentionally taking an injection from the same pen twice. Thus an alarm is not transmitted within the precautionary time.

The invention is not limited solely to tubes, but the sockets can be composed of one or several longitudinal supporting elements with another shape, said supporting elements extending in the height direction. It is for example possible to use several rib-like elements around the dosage unit 3. One supporting element with an open cross-section may also be sufficient for each socket, said supporting element being located in a sloping position in such a manner that the dosage unit 3 rests against the same and said supporting element may thus be shaped on the side of the dosage unit as a curved groove-like structure. In the alternatives where the supporting element does not surround the dosage unit 3 entirely as a tube-shaped element, the dosage unit can be removed without a lifter as well, but also in these cases it is possible to arrange a mechanical lifting movement in conjugation with the time of administering the dose, wherein, in addition to the visual indicator, said dosage unit 3 is at the same time distinguishable from the others. Also in this case the supporting elements can be dimensioned in such a manner that in the normal position the upper ends of the dosage units remain below the upper end of the supporting element, and the lifter 4 lifts the upper end of the dosage unit 3 above the upper end of the element.

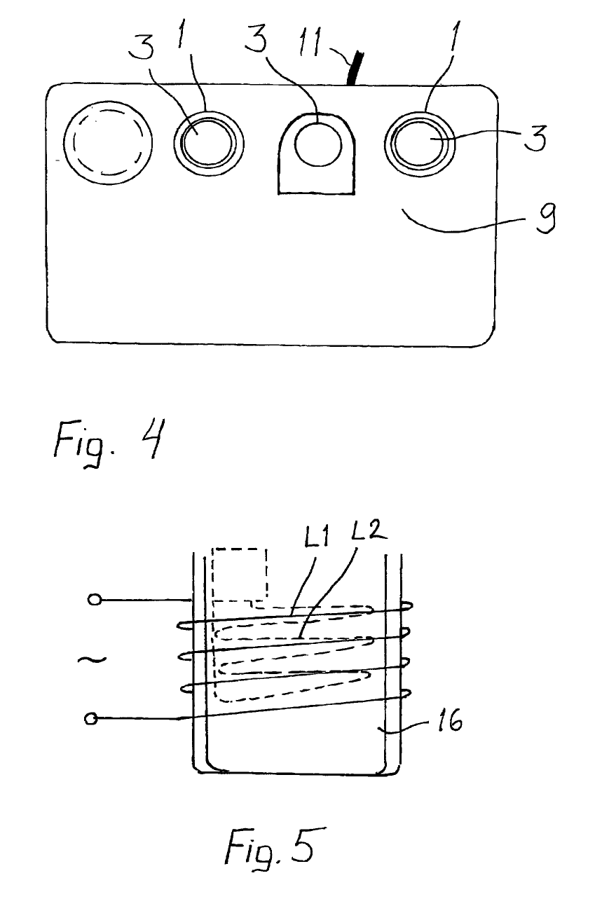

FIG. 4 shows that all tubes or sockets 1 with another shape do not necessarily have to contain an injection pen, but the device can be programmed for a smaller number of different injection pens. The basic model is a device with four sockets, wherein the maximum number of injection pens is four. The connecting cable by means of which the device can be connected to a plug socket is marked with the reference numeral 11. In FIG. 4 one of the sockets is missing (a tube or a corresponding supporting element/elements has/have been released) and the hole remaining in the body casing is plugged. One of the sockets is also modified into a special seat for a portable injection pen provided with a control device of its own, containing a sensor detecting the movement relating to the injection (e.g. the movement of the piston or a part connected thereto kinetically). Thus, the seat functions as a charger for the battery of the control device. The control device can be the device presented in the international publication WO99/43283 that can be attached to the injection pen as a separate element and contains timer and alarm functions of its own. When the body casing is designed, such a charger possibility can be taken into account for example in such a manner that one hole in the body casing, in which the lower end of the dosage unit is located in normal use, is equipped with current contacts, for example contacts supplying direct current, which are otherwise covered by the tube fixed to the hole e.g. by means of screwing down, but which are coupled to the conductors of a fitting element attached in the hole to replace the tube, said conductors passing to the charging contacts of the fitting element. Into such a fitting element fixed to the body casing it is now possible to insert a control device accompanying the pen, the contacts of the control device being now connected to the contacts of the fitting element. Another structural possibility is to arrange a hole in the body casing which is originally wider and functioning as a charger and containing current contacts required for the charging in readiness, and the control device can be inserted in the hole. When the aim is to change this section into a conventional socket, a fitting element is inserted therein, containing an attachment point for a tube or another supporting element, or a tube or a corresponding supporting element is already contained therein.

In the charging by means of the body casing, the control device and the injection pen are attached to each other, wherein they can be easily taken along from the body casing. Naturally, it is also possible to register the placement of the pen and the control device in the charging and release from the charging into the memory of the device, because such a movement can be easily detected and this information can also be read from outside by means of the data transmission line. FIG. 4 shows how among three “operating” sections the one in the middle functions as a charger for the control device of a portable injection pen. In the treatment of diabetes it is thus possible to act in such a manner that the morning insulin is taken at home from the injection pen of the outermost socket, the portable injection pen and the control device are taken along from the charger when going away for the day, and the evening insulin is again taken at home from the other outermost socket. When the body casing is modified, the movements of the lifters 4 are of course programmed to occur according to the locations of those injection pens that can be lifted up. When eccentrics located on the same shaft are used, the shaft 8 can revolve double a distance at the moment of operation, so that it is possible to pass by the empty (plugged) section or the section functioning as a charger. If the diabetic is at home at all times when a dose must be taken, in the place of the portable injection pen and the charger of its control device there is a socket similar to those intended for other injection pens.

The same principle can be applied for four-injection treatment, wherein the plugged hole seen in the drawing also provides an operative development location. Thus, the order can be such that on the right-hand side there is the socket for the injection pen containing the morning insulin, the following socket is for the portable injection pen for the daytime insulin to be taken before lunch, and it is followed by the socket for the injection pen for insulin to be taken around at 5 p.m., and on the left-hand side there is a socket for the “night insulin” to be taken late in the evening. When necessary, the device can be designed in such a manner that the chronological order of the sockets and charger locations proceeds from left to right. If the diabetic is at home at all times a dose must be taken, in the place of the portable injection pen and the charger of its control device there is a socket similar to those intended for other injection pens.

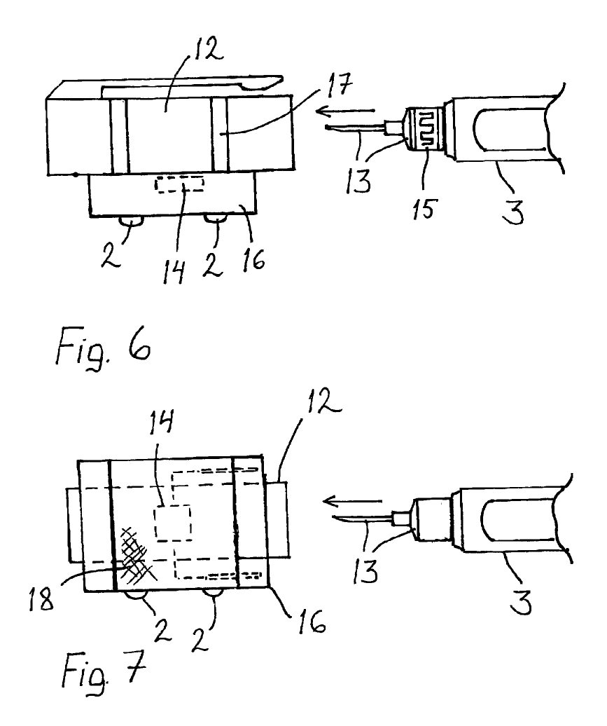

FIG. 5 shows a preferred embodiment of charging the control device of a portable injection pen, which can be applied in the device shown hereinabove in FIG. 4. The charging functions inductively, wherein the table control device contains a primary coil L1 which is connected to a power source and functions as a charger, and surrounds the charger socket 1 to which the portable injection pen is placed. The injection pen may be fastened into its control device, or the injection pen can also be of the type to which the control device of its own is integrated in such a manner that it cannot be released from the injection pen. The control device on the injection pen is positioned inside the charger socket, and it contains a secondary coil L2 connected via a rectifier to a rechargeable battery functioning as a power source, said secondary coil being positioned inside the coil in the charger socket 1, and having charging current induced thereto as a result of the alternating current in the primary coil L1 of the charger socket. The primary socket L1 and the secondary socket L2 may also be positioned in another way with respect to each other in the charging position, so that they are inductively coupled to each other.

At the same time when the control device of the portable injection pen is set into a charging position, it is possible to transfer information thereto from the table control device, and the table control device may read information contained therein. The data transmission may take place via a separate line, but advantageously charging current is also used for data transmission, wherein the charging current contains components which the control device of the portable injection pen recognizes as data and transfers to the memory. The information contained in the control device of the portable injection pen can be transferred therefrom to the table control device, i.e. it can be “discharged” via the same line by means of which the charging is conducted, for example after the charging, when there is certainly a sufficient amount of power available for data transmission. Combined charging and data transmission can be implemented inductively or with a direct charging current contact. Concerning the principle of inductive charging and simultaneous change of data between the charged device and charger device, reference is made to U.S. Pat. Nos. 6,028,413 and 5,455,466, respectively. The disclosures of these two documents are incorporated herein by reference.

Exchange of information between the control device of the portable injection pen and the table control device can take place in another wireless manner, for example by means of an infrared connection.

FIG. 6 shows the control device 16, which is incorporated in the portable injection pen especially for prevention of a double injection and whose power source (battery) can be charged by the table control device in the manner described hereinabove. The device is based on the idea that when a dose is taken, the cap 12 protecting the needle must always be removed. In the body of the injection pen 3, for example at the base of the needle part 13 there is a resonance circuit 15 (a so-called passive tag). An active transmitter-receiver part 14 connected to the power source (rechargeable battery of the control device) that generates a radio field is located in the cap 12. The principle is similar to the one used in shop control gates, but in a sort of a way in reversed order, i.e. in the normal situation (the passive tag and the active part located close to each other, i.e. the cap 12 on top of the needle 13), the resonance circuit 15 is located in the radio field (in which the shop control gate would give an alarm). When the resonance circuit 15 is not located close to the active part 14, a different kind of radio field is detected by the active part. In this situation, in which shop control gates are in the normal situation, we are dealing with a special case, i.e. the cap 12 has been removed from the top of the needle 13. When the cap is removed, the control device detects the removal of the cap through the missing resonance circuit. The resonance circuit 15 can be arranged in the cylindrical plastic part located at the base of the needle part 13, for example by attaching an adhesive tape containing the circuit to the part or embedding the circuit 15 in this part already at the manufacturing stage. It is, for example, possible to produce a ring-like or cylindrical part that fits tightly around the plastic part and is made of an adhesive film containing the circuit. This part can be removed from the plastic part when a needle 13 is changed in the injection pen 3, or the entire injection pen is replaced. The principle of recognizing an event of administration of a dose is otherwise the same as the one described above. The cap 12 must be removed from the top of the needle 13 for a period of fixed length. In practice, the time is measured in such a manner that to save power the active transmitter-receiver part 14 first checks, for example within short intervals (1 to 2 s), whether the resonance circuit 15 is present, and when a fixed number of successive checking steps have been calculated in which the resonance circuit is missing, the interpretation is made that the injection has been taken. The actual control device 16 containing a transmitter part, a processor, a timer and a power source is a separate piece that can be fixed to a conventional cap 12 of an injection pen 3, and it contains an indicator 2. When it is time to take a dose from the pen, the visual indicator 2 indicates that a dose should be taken. At the same time it is possible to give an acoustic alarm. The release of the cap 12 is recognized on the basis of the above-described principle based on the resonance circuit 15. When the event of administration of the dose has been registered in this way, the visual indicator 2 by the pen remains, for a given time, for example for a set precautionary time (for example 2 to 3 hours), in a state that shows that the dose has already been taken from the pen. This is indicated advantageously with a colour light that can be easily detected. The indicator 2 showing that a dose should be taken and the indicator showing that the dose has been taken can be different indicators which are turned “on” and “off”, for example implemented with LEDs of different colours (for example green: take a dose, red: a dose taken). It is, of course, possible to use physically the same indicator which changes its state, e.g. colour, according to the state of the pen in question. If an attempt is made to take a dose again within the precautionary time, the control device gives an alarm (a special warning signal) on the event of administration of a double injection, for example by means of an acoustic signal (signal tone). The control device does not, however, prevent one from taking a second injection within the precautionary time. When an administration time and a precautionary time of a fixed length after the event of administration (measured with a timer) is not on in the control device, the indicators of the device are in the passive state (for example the visual light-emitting indicator/indicators are turned off). Alternatively, to save the power source it is possible to keep the light/lights in a turned-off state when the dose has been taken appropriately, and turn them on only when the cap is removed for the second time during this precautionary time, wherein the lit light is a warning (an acoustic signal can also be given).

In its simplest form such double injection warning device in a portable pen can only contain the aforementioned resonance circuit 15 and the active part 14 arranged in the cap 12, as well as the indicator/indicators 2, and it only requires such an amount of data processing and time measuring capacity and such a power source that the device is capable of registering the dose as administered. When the device is within a fixed precautionary time in the state “dose taken”, the indicator gives an alarm if the sensors detect the removal of the cap. This precautionary time could, in principle, be set so that it would end only at the time of administration of the next injection, but it is advantageously shorter, so that the pen can be serviced every now and then without causing an alarm.

The control device 16 can be attached to the existing cap 12 of an injection pen by means of one or several fixing means that can be attached around the cylindrical portion of the cap 12 and are designated at 17 in the figure. Alternatively, the control device 16 can be formed as a bushing that has a cavity wherein the cap 12 can be fitted with a suitable tightness so that the control device 16 surrounds the cap 12. This general configuration is depicted in FIG. 7.

FIG. 7 shows an embodiment where the control device 16 cooperates with the needle 13 without a complementary sensor in the needle part 13 or elsewhere in the injection pen 3. The control device 16 contains a sensor 14 that acts on capacitive principle, that is, the sensor 14 has two electrodes that form a capacitor. The change of the properties of an RC-circuit being part of the sensor can be detected in the form of a changed natural oscillation frequency. The presence or absence of the needle part 13 or the proximal end of the injection pen body between the electrodes can be detected and the the administration of a dose can be deduced analogously to the previous example of FIG. 6. In fact when any matter is in the vicinity of the capacitive sensor 14, it affects its capacitance and consequently the properties of the sensor circuit that can be easily detected.

The capacitive sensor 14 in the cap 12 can be utilized even in a more sophisticated manner. When sensitive enough, it can be used to detect the change of the insulin volume of the insulin container in the injection pen 3. This requires the measurement before and after the injection in exactly the same position of the cap 12, that is, while it is over the needle 13. A sensor detecting a grip by the fingers can be incorporated in the control device 16 in this case, and it can be a sensitive sensor detecting the presence of human fingers through the electrical conductivity of skin, heat by the skin, or pressure applied by the fingers, and solutions known from various control interfaces of a human and a device reacting to just a light touch by a finger can be applied. One example of a sensor film having an efficient response to a pressure is the electromechanical film widely known as EMF. The sensor is designated at 18 in FIG. 7, and it can be placed around the device 16 along its periphery over an area where the device 16 is gripped by fingers when the cap 12 is to be removed. When the sensor 18 of any aforementioned kind detects a grip, it immediately gives a command to the capacitive sensor 14 to perform a measurement. When the removal of the cap 12 has been detected, and the cap 12 thereafter is back on its place, the measurement data just before the cap removal and the measurement data of the normal situation of the cap being back again is compared, and a slight difference of the readings will indicate that the container volume has decreased, i.e. the insulin has flown from the container through the needle. This difference of measurement values is smaller than the difference of the values between the situations where the cap 12 is over the needle 13 on the pen and away from the pen, respectively. This arrangement of combining another suppplementary measurement with the actual detection measurement of the cap removal may be helpful in deciding whether the cap 12 has been just removed for a while and put back, or whether in fact some insulin has been injected. To work in a reliable manner the electrodes of the capacitive sensor 14 in the control device 16 should be close to the body of the injection pen 3 and protected aginst external influences by a surrounding shield so that the the parts of the insulin pen 3 closest to the needle part 13 (insulin container) have the largest influence on the capacitance of the sensor 14 and the changes experienced by the sensor.

Above the embodiments have been described where the control devices can be fitted to ordinary, usually cylindrical caps of commercially available existing injection pens. It is also possible to produce a special cap, in which the control device is integrated so that it forms an inseparable single unit with the cap. The special cap can be changed in an injection pen to replace a conventional cap at the same time when a resonance circuit is placed at the base of the needle. This specially designed cap can use all sensor embodiments presented hereinabove.

The control device 16 placed on or integrated in the cap can be placed in the charging socket of the table control device, and the charging and data transmission can be implemented according to the above-described non-contacting and contacting principles.

Furthermore, when the needle part 13 also has a sensor, such as the circuit 15 discussed above, it is possible to produce separately needles in which the sensor part required by the control device 16 has already been integrated. This sensor part, such as the aforementioned resonance circuit, can be placed in the wider part located at the base of the needle, for example inside this part or on top of the same.

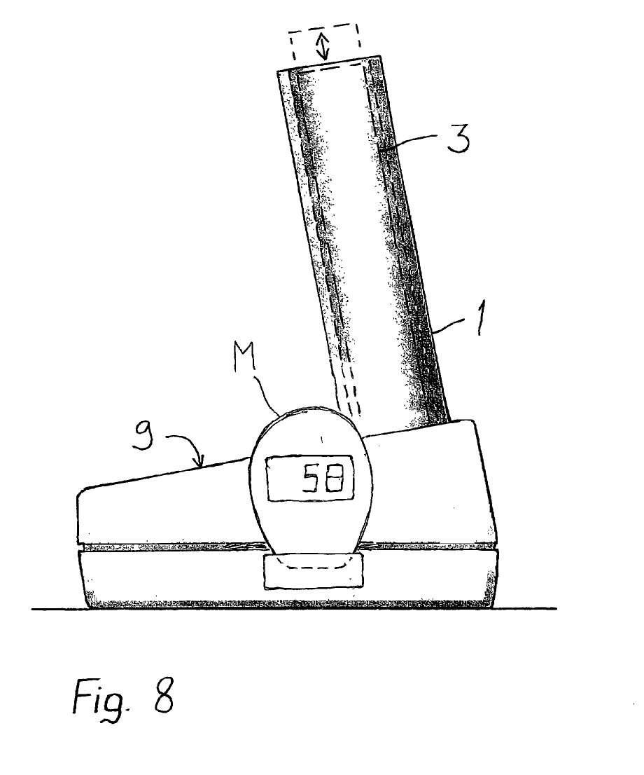

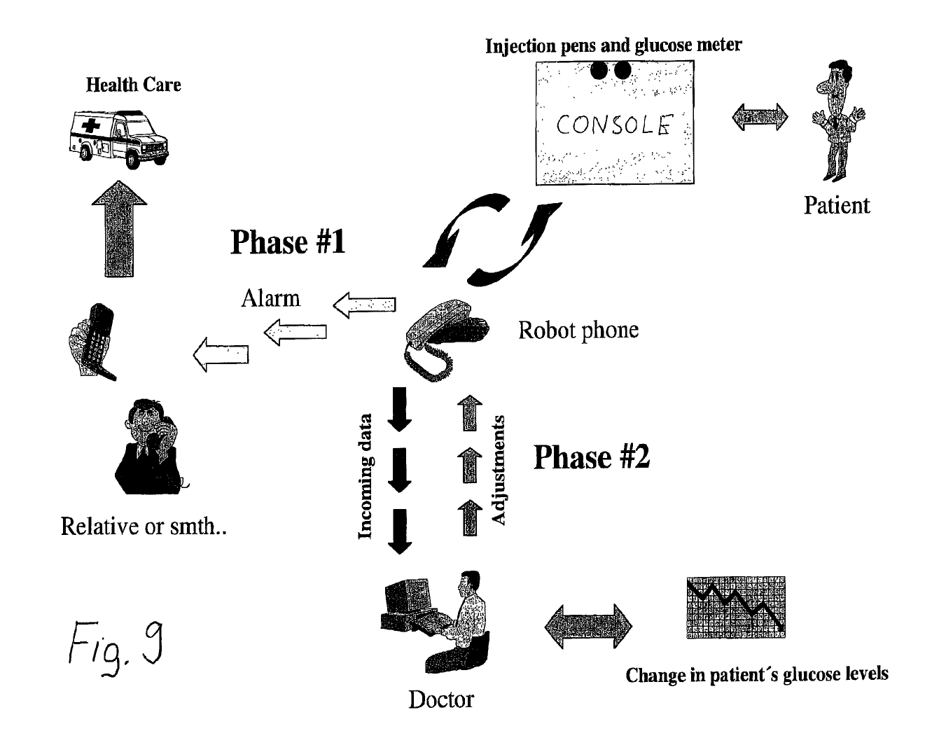

FIG. 8 shows how it is possible to use the table control device also for monitoring other kind of treatment related to diabetes. A portable blood sugar measurement device M, which by means of an analysis principle known as such measures the blood sugar content in the blood sample placed therein, can be placed in the control device. The blood sugar measuring device contains information on the measuring moment and the measured blood sugar, and the memory of the same may contain blood sugar information measured at different times. The measured data and measuring times are stored in the memory of the table control device by means of a data transmission connection between the measurement device and the control device, for example by means of interface points based on mechanical contact, and this information can also be transmitted to an outside supervisor via a data transmission line.

FIG. 9 shows schematically the data transmission system, according to which the information contained in the table control device “CONSOLE”, including blood sugar measurement information, are read automatically at fixed intervals, preferably once a day, for example at night. According to the way shown in FIG. 8, the table control device can be connected to a so-called robot phone. The robot phone may be integrated in the table control device, wherein it can be implemented with a card containing the necessary electronics. The table control device can also be provided with a GSM card, wherein the data transmission takes place in a wireless manner. The drawing shows how the transmission of information outside from the control device can take place via a public telecommunication network or a wireless data transmission connection in two ways. The first way, marked with “Phase#1” illustrates the above-mentioned alarm cases. The second way, marked with “Phase#2” illustrates at least data transmission outside from the device at fixed intervals (“incoming data”) by means of which at least information on the times of administration of doses and advantageously also on the blood sugar values is transmitted to an outside expert (“Doctor”) automatically at fixed times of the day, preferably at night time. The treatment actions and the results of the same from the previous day (Change in patient's glucose levels) can thus be read by the expert for example on a computer screen first thing in the morning, and on the basis of this data it is possible to use the same line to transmit information back to the table control device (instructions and changes in values, marked with the word “Adjustments”). Thus, this data transmission is preferably bi-directional.

I claim:

1. A portable control device for monitoring the administration of doses by a diabetic and arranged in conjunction with a portable dosage unit having a body and an injection member designed to deliver the dose to the diabetic and attached to the body of the portable dosage unit, and a removable cap over the injection member, the control device being provided in the cap and being movable together with the cap, said control device comprising a sensor arranged to detect the removal of the cap from over the injection member.

2. The portable control device according to claim 1, wherein a sensor part is placed in the injection member or at the proximal end of a body of the dosage unit, and arranged in cooperation with the sensor of the control device.

3. The portable control device according to claim 2, wherein the sensor part placed in the injection member is a resonance circuit.

4. The portable control device according to claim 1, wherein said control device provided in the cap comprises a sensor acting on capacitive principle.

5. The portable control device according to claim 1, wherein said control device provided in the cap comprises at least one indicator related to the administration of the dose.

6. The portable control device according to claim 5, wherein the indicator is connected to the sensor and arranged to indicate the removal of the cap from over the injection member.

7. The portable control device according to claim 5, wherein the indicator is arranged to stay for a predetermined period of time in a state independent of the position of the cap during said predetermined period of time, the state indicating the removal of the cap from over the injection member.

8. The portable control device according to claim 7, wherein the indicator is arranged to indicate the removal of the cap from over the injection member only in the case where a predetermined delay time has elapsed from the removal of the cap from over the injection member.

9. The portable control device according to claim 5, wherein the indicator is connected to a time measuring device and is arranged to indicate to the diabetic a pre-programmed time of administering the dose measured by the time measuring device.

10. The portable control device according to claim 1, wherein the control device provided in the cap is a separate body mountable on the cap.

11. The portable control device according to claim 10, wherein the separate body comprises one or several fixing means attachable around the cap.

12. The portable control device according to claim 10, wherein the separate body is a bushing-like body arranged to fit around the cap.

13. Table control device for monitoring the administration of doses by a diabetic, comprising one or more places for a removable and portable dosage unit containing an agent intended for treatment of diabetes and comprising a cap removable from the dosage unit, at least one dosage unit comprising an electronical control device provided in the cap, the table control device comprising further one or several indicators, a memory for storing data, and a charger for the electronical control device provided in the cap of the dosage unit.

14. The table control device according to claim 13, wherein the charger is an inductive charger.

15. The table control device according to claim 13, wherein the charger is arranged to transmit data between the table control device and the electronic control device provided in the cap.

16. The table control device according to claim 13, wherein the table control device is connected through a data transmission line to a remote monitoring location.

17. The table control device according to claim 15, wherein the table control device is connected through a data transmission line to a remote monitoring location.

18. The table control device according to claim 16, wherein the data transmission line is bidirectional.

19. The table control device according to claim 17, wherein the data transmission line is bidirectional.

20. Table control device for monitoring the administration of doses by a diabetic, comprising one or more places for a removable and portable dosage unit containing an agent intended for treatment of diabetes and comprising a cap removable from the dosage unit, the table control device comprising further one or several indicators, a memory for storing data, a place for accommodating a portable blood sugar measurement device, and a data transmission connection between the portable blood sugar measurement device and the memory of the table control device.

21. The table control device according to claim 20, wherein the table control device is connected through a data transmission line to a remote monitoring location.

22. The table control device according to claim 21, wherein the data transmission line is bidirectional.
